(19) United States
(12) Patent Application Publication
(10) Pub. No.: US 2008/0004694 A1
(43) Pub. Date: Jan. 3, 2008

(54) COMPOSITIONS AND METHODS FOR ADMINISTERING DEXAMETHASONE WHICH PROMOTE HUMAN CORONARY ARTERY ENDOTHELIAL CELL MIGRATION

(75) Inventors: Matthew Mack, Chicago, IL (US); Sandra Burke, Libertyville, IL (US); John Toner, Libertyville, IL (US); Keith Cromack, Gurnee, IL (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/752,093

(22) Filed: May 22, 2007

Related U.S. Application Data
(60) Provisional application No. 60/802,728, filed on May 23, 2006.

(51) Int. Cl. A61F 2/06

(52) U.S. Cl. 623/1.42

(57) ABSTRACT

A drug-eluting endoprosthesis is configured for inhibiting restenosis and thrombosis, and for promoting healing of a lesion in a body of a subject. Such an endoprosthesis includes at least a supporting structure and a therapeutically effective amount of dexamethasone is disposed thereon. The therapeutically effective amount of dexamethasone allows for the drug to elute from the supporting structure so as to obtain a concentration of dexamethasone that is sufficient for inhibiting restenosis and/or inhibiting thrombosis. Also, the therapeutically effective amount of dexamethasone is substantially devoid of inhibiting cell migration such that migrating cells promote healing of the lesion. Additionally, the therapeutically effective amount of dexamethasone induces cell migration such that migrating cells promote healing of the lesion.

### CROSS-REFERENCE TO RELATED APPLICATIONS

This U.S. patent application claims benefit of U.S. provisional patent application having Ser. No. 60/802,728, filed on May 23, 2006, entitled “COMPOSITIONS AND METHODS OF ADMINISTERING DEXAMETHASONE WHICH PROMOTES HUMAN CORONARY ARTERY ENDOTHELIAL CELL MIGRATION,” and having Matthew Mack, Sandra Burke, John Toner, and Keith Cromack as inventors, which U.S. provisional patent application is incorporated herein in its entirety by specific reference.

### BACKGROUND OF THE INVENTION

I. The Field of the Invention

The present invention relates to systems, medical devices, and methods for delivering dexamethasone so as to not substantially inhibit human coronary artery endothelial cell migration. More particularly, the present invention relates to systems, medical devices, and methods that include the use of an endoprosthesis, such as a stent, to deliver dexamethasone in a manner that promotes human coronary artery endothelial cell migration.

II. The Related Technology

Stents, grafts, and a variety of other endoprostheses are well known and used in interventional procedures, such as for treating aneurysms, for lining or repairing vessel walls, for filtering or controlling fluid flow, and for expanding or scaffolding occluded or collapsed vessels. Such endoprostheses can be delivered and used in virtually any accessible body lumen of a human or animal, and can be deployed by any of a variety of recognized methodologies. One recognized indication of an endoprosthesis, such as a stent, is for the treatment of atherosclerotic stenosis in blood vessels; however, stents are used to treat a variety of maladies associated with blood vessels and other lumen within the body. For example, after a patient undergoes a percutaneous transluminal coronary angioplasty or other similar interventional procedure, a stent is often deployed at the treatment site to improve the results of the medical procedure and reduce the likelihood of restenosis. However, the placement of a stent in a blood vessel may injure the vessel and cause lesions in the walls of the vessel.

Mechanical injury induced by stent implantation can cause endothelial denudation, which is directly associated with the formation of lesions in the vessel wall. The formation of lesions in the blood vessel wall can initiate an inflammatory response within the vasculature wall of a blood vessel. As such, this can cause the activation of circulating platelets, the infiltration of neutrophils and monocytes, and the release of pro-inflammatory cytokines and growth factors. Inflammation is a major stimulus for alteration of smooth muscle cell phenotype, and can result in smooth muscle cell activation, proliferation, and migration into the neointima, which causes restenosis. Also, recent studies suggest that such alterations in smooth muscle cell phenotype may be a result of smooth muscle cell de-differentiation into a myofibroblast phenotype. Thus, the physiological response to the mechanical injury caused by a stent can induce restenosis.

Additionally, mechanical injury induced by stent implantation may also cause proliferation and migration of vascular endothelial cells. The proliferation and migration of vascular endothelial cells can induce the re-endothelialization of the stented blood vessel so as to reduce lesion thrombosis. In instances that lesions in vessel wall are not re-endothelialized, lesion thrombosis can occur, which is problematic. As such, there is a need to reduce restenosis and thrombosis after stent implantation.

It has been found that some drug-coated stents decrease the risk of stent-induced restenosis by inhibiting the proliferative response associated with endothelial denudation. It is thought that some drugs that inhibit restenosis bind to cytosolic FKBP-12 and inhibit the protein kinase mTOR. The mTOR kinase may be involved in cell cycle progression by altering phosphorylation of downstream targets such as p70S6 kinase (p70S6K). As such, these drugs can inhibit p70S6K phosphorylation, which can inhibit endothelial cell proliferation.

While drug-eluting stents may provide favorable responses to inhibit restenosis, the drugs eluted from the stent may also lead to thrombosis. In part, this may be because the drug inhibits endothelial cell migration, which in turn inhibits the re-endothelialization of lesions, thereby leading to thrombosis. As such, there is a need for a drug-eluting stent that does not inhibit the re-endothelialization of lesions that are caused from implantation of the stent. Thus, there is a need for a drug-eluting stent that is balanced between inhibiting restenosis while permitting re-endothelialization of lesions, and thereby inhibiting thrombosis.

Therefore, it would be advantageous to have an endoprosthesis and method of use thereof that inhibits restenosis and thrombosis. Also, it would be advantageous to have an endoprosthesis and method of use thereof that inhibits p70S6K phosphorylation, and thereby inhibits cell proliferation, but allows and/or promotes for endothelial cell migration to re-endothelialize lesions in the vessel wall so as to inhibit thrombosis.

### BRIEF SUMMARY OF THE INVENTION

The present invention generally includes endoprostheses, deployment systems, and methods for delivering dexamethasone from an endoprosthesis in an amount that can inhibit restenosis and does not substantially inhibit cell migration adjacent or proximal to the endoprosthesis. More particularly, the present invention includes the use of an endoprosthesis, such as a stent, to deliver dexamethasone that so as to promote cell migration. For example, when the endoprosthesis is a stent configured for being deployed within a human coronary artery, dexamethasone can inhibit restenosis of the coronary artery without substantially inhibiting coronary artery cell migration adjacent or proximal to the stent and can even promote the migration of cells. Moreover, dexamethasone can be eluted in an amount that promotes the migration of endothelial cells and/or smooth muscle cells, which can allow for re-endothelialization of a lesion that may be formed in a wall of the vessel from the deployment of the endoprosthesis.

In one embodiment, the present invention includes a drug-eluting endoprosthesis configured for inhibiting restenosis and for promoting healing of a lesion in a body of a subject. Such an endoprosthesis includes at least a supporting structure and dexamethasone disposed on the supporting structure. The supporting structure is configured and dimensioned to be placed in the body of the subject, such as a body lumen. A therapeutically effective amount of dexamethasone is disposed on the supporting structure. The therapeutically effective amount of dexamethasone allows for the drug to elute from the supporting structure so as to obtain a concentration of dexamethasone in the body that is sufficient for inhibiting restenosis and that is substantially devoid of inhibiting cell migration adjacent to the supporting structure when disposed within the subject. Accordingly, by not substantially inhibiting cell migration, dexamethasone can allow the migrating cells to promote healing of the lesion. Optionally, the lesion is in a body lumen selected from the group consisting of a blood vessel, artery, coronary artery, vein, esophageal lumen, and urethra.

Additionally, the therapeutically effective amount of dexamethasone can allow for the drug to elute from the supporting structure so as to obtain a concentration of dexamethasone in the body that is sufficient for promoting cell migration adjacent to the supporting structure when disposed within the subject. Accordingly, by promoting cell migration, dexamethasone can induce the migration of cells to promote healing of the lesion.

In one embodiment, the dexamethasone can be any pharmaceutically acceptable derivatives, analogs, salts, prodrugs, or esters thereof

In one embodiment, the endoprosthesis includes a pharmaceutically acceptable carrier containing dexamethasone disposed on the supporting structure.

In one embodiment, the endoprosthesis includes a coating containing dexamethasone being disposed on the supporting structure. Optionally, the coating is a biocompatible polymer. In another option, the coating controls the rate dexamethasone is eluted from the supporting structure.

In one embodiment, the dexamethasone is present on the supporting structure in an amount of 10 ng/mm to about 10 mg/mm of length of the endoprosthesis.

In one embodiment, the dexamethasone is present on the supporting structure in a concentration from about 10 ng/ml to about 10 mg/ml.

In one embodiment, the local concentration of dexamethasone eluted from the endoprosthesis is sufficient for inhibiting restenosis. Also, the local concentration does not substantially inhibit cell migration adjacent to the supporting structure when disposed within the subject. Additionally, the local concentration of dexamethasone can promote cell migration adjacent or proximal to the supporting structure so as to promote re-endothelialization of any lesions. Such a local concentration is from about 10 pg/ml to about 10 mg/ml.

In one embodiment, the dexamethasone elutes from the supporting structure at a rate of about 10 pg/day to about 10 ug/day.

In one embodiment, the present invention includes a method of inhibiting restenosis and promoting healing of a lesion in a body of a subject. Such a method includes deploying an endoprosthesis that contains dexamethasone into the body of the subject. The endoprosthesis includes a supporting structure configured and dimensioned to be placed in the body of the subject. A therapeutically effective amount of dexamethasone is disposed on the supporting structure. The dexamethasone is eluted from the supporting structure so as to obtain a concentration of dexamethasone in the body and adjacent or proximal to the supporting structure that is sufficient for inhibiting restenosis and that is substantially devoid of inhibiting cell migration adjacent or proximal to the supporting structure when disposed within the subject. Accordingly, by not substantially inhibiting cell migration, dexamethasone can allow the migrating cells to promote healing of the lesion. Additionally, the dexamethasone can induce the migration of cells to promote healing of the lesion Optionally, the lesion is in a body lumen selected from the group consisting of a blood vessel, artery, coronary artery, vein, esophageal lumen, and urethra.

In one embodiment, the method includes eluting dexamethasone from a pharmaceutically acceptable carrier disposed on the supporting structure.

In one embodiment, the method includes eluting dexamethasone from a coating disposed on the supporting structure.

In one embodiment, the method includes achieving a local concentration of dexamethasone from about 10 pg/ml to about 10 mg/ml. The local concentration can be within a tissue, cell, or fluid adjacent to the endoprosthesis.

In one embodiment, the method includes eluting dexamethasone from the supporting structure to achieve a substantially steady-state concentration of about 10 pM to about 10 uM.

In one embodiment, the method includes inhibiting restenosis of a body lumen adjacent to the supporting structure. This can also be performed while promoting healing of the lesion in a wall of the body lumen that is caused by the supporting structure. The healing of the lesion is promoted by allowing and/or promoting the migration of endothelial cells to the lesion. Such cell migration can lead to re-endothelialization of the lesion.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a graph showing the effects of zotarolimus, sirolimus, dexamethasone, and paclitaxel on hCaEC migration following acute drug treatment. Cells were synchronized for 24 hours prior to the addition of drug. Cells were not pretreated prior to the induction of migration.

FIG. 1B is a graph illustrating the effect of zotarolimus, sirolimus, dexamethasone, and paclitaxel on inhibiting migration of human coronary artery smooth muscle cells following acute drug treatment. Cells were synchronized for 24 hours prior to the addition of drug. Cells were not pretreated prior to the induction of migration.

FIG. 1C is a graph illustrating the IC₅₀ of the inhibitory effects of paclitaxel on the migration of human coronary artery smooth muscle cells and human coronary artery endothelial cells following acute drug exposure.

FIG. 2 is a graph illustrating the effects of pretreating human coronary artery endothelial cells with the mTOR antagonists (zotarolimus, sirolimus, and everolimus), dexamethasone, and paclitaxel on the inhibition of human coronary artery endothelial cell migration. hCaEC were synchronized and then treated for 24 hours in hCaEC growth media containing drugs. Migration was then assessed in response to hCaEC growth media (i.e. media containing 5% FBS and growth factors). Cells were resuspended in basal media containing drug during migration.

FIG. 3 is a graph illustrating the effect of different concentrations of zotarolimus, dexamethasone, sirolumus, and paclitaxel on FBS-induced human coronary artery endothelial cell migration.

FIG. 4 is a graph illustrating the effect of zotarolimus, dexamethasone, sirolumus, and paclitaxel on FBS-induced human coronary artery smooth muscle cell migration.

FIG. 5 is a graph illustrating the effect of zotarolimus, dexamethasone, and paclitaxel on PDGF-BB stimulated human coronary artery smooth muscle cell migration.

FIG. 6A is a depiction of a western blot that shows the effect of various substances on p70S6K (T389) phosphorylation in human coronary artery endothelial cells.

FIG. 6B is a graph illustrating the densitometry of the western blots from human coronary endothelial cells.

FIG. 7 is a depiction of a western blot that shows the effect of various substances on p70S6K (T389) phosphorylation in human coronary artery smooth muscle cells.

### DETAILED DESCRIPTION OF THE PREFERRED EMBODIMENTS

The present invention generally includes endoprostheses, deployment systems, and methods for delivering dexamethasone in an amount that can inhibit restenosis and does not substantially inhibit cell migration adjacent to the endoprosthesis. More particularly, the present invention includes the use of an endoprosthesis, such as a stent, to deliver dexamethasone that does not substantially inhibit cell migration adjacent to the deployed endoprosthesis. For example, when the endoprosthesis is a stent that is deployed within a human coronary artery, dexamethasone can inhibit restenosis of the coronary artery without substantially inhibiting coronary artery cell migration. Moreover, dexamethasone can be eluted in an amount that does not inhibit the migration of endothelial cells and/or smooth muscle cells, which can allow for re-endothelialization of a lesion that may be formed from deployment of the endoprosthesis. Thus, the present invention includes endoprostheses, deployment systems, and methods that inhibit restenosis and thrombosis.

Additionally, the dexamethasone can promote migration of cells. For example, when the endoprosthesis is a stent that is deployed within a human coronary artery, dexamethasone can inhibit restenosis of the coronary artery and promote coronary artery endothelial cell migration. Moreover, dexamethasone can be eluted in an amount that induces or promotes migration of endothelial cells and/or smooth muscle cells, which can allow for re-endothelialization of a lesion that may be formed from deployment of the endoprosthesis. Thus, the present invention includes endoprostheses, deployment systems, and methods that inhibit restenosis and thrombosis by administering an amount of dexamethasone that promotes migration of cells within a vessel.

I. Introduction

In one embodiment, the present invention includes endoprostheses, deployment systems, and methods for delivering an amount of dexamethasone that inhibits restenosis and thrombosis. As such, the present invention includes endoprostheses, deployment systems, and methods that include the use of an endoprosthesis, such as a stent, to deliver dexamethasone in a manner that promotes human coronary artery endothelial and/or smooth muscle cell migration. Thus, the present invention relates to endoprostheses, deployment systems, and methods for inhibiting restenosis and thrombosis without inhibiting endothelial and/or smooth muscle cell migration or by promoting the migration of endothelial cells.

While the present invention is described in connection with drug-eluting stents that can be placed within the human coronary artery, the systems and methods of the present invention are applicable to other blood vessels or other lumen passageways within the body. A drug-eluting endoprosthesis in accordance with the present invention can be used in applications to inhibit restenosis and to induce cell migration in order to promote re-endothelialization of lesions so as to inhibit thrombosis.

Accordingly, the drug-eluting endoprosthesis in accordance with the present invention can be used in the treatment and/or prevention of hyperproliferative vascular diseases such as intimal smooth muscle cell hyperplasia, restenosis, and vascular occlusion without substantially increasing susceptibility to thrombosis. Such hyperproliferative vascular diseases may occur following biologically- or mechanically-mediated vascular injury, and can be treated or prevented by use of the drug-eluting endoprosthesis as described herein without causing thrombosis.

II. Drug Delivery System

In one embodiment, the present invention includes a drug delivery system that has a supporting structure with a therapeutically effective amount of dexamethasone described herein. The therapeutically effective amount of dexamethasone is an amount that does not substantially inhibit cell migration adjacent or proximal to the deployed supporting structure. For example, the therapeutically effective amount of dexamethasone does not substantially inhibit human coronary artery endothelial and/or smooth muscle cell migration.

In one embodiment, the therapeutically effective amount of dexamethasone is an amount that inhibits restenosis without substantially inhibiting cell migration adjacent or proximal to the deployed supporting structure. For example, the therapeutically effective amount of dexamethasone inhibits restenosis, but does not substantially inhibit human coronary artery endothelial and/or smooth muscle cell migration.

Additionally, the therapeutically effective amount of dexamethasone is an amount that induces cell migration adjacent or proximal to the deployed supporting structure. For example, the therapeutically effective amount of dexamethasone induces human coronary artery endothelial and/or smooth muscle cell migration.

The supporting structure can be configured from a wide variety of materials and in a wide variety of configurations. The supporting structure can be a part of a medical device, such as an endoprosthesis, that is employed within a body of a patient. Examples of endoprostheses can be stents, coronary stents, peripheral stents, grafts, arterio-venous grafts, bypass grafts, drug delivery balloons, drug delivery depots, catheters, pick lines, valves, and the like. The materials that the medical devices are made of can include biocompatible materials such as polymers, plastics, metals, ceramics, and other materials that can be utilized within the body of a patient.

In one embodiment, the supporting structure can be comprised of a shaped memory material (“SMM”). For example, the SMM can be shaped in a manner that allows for restriction to induce a substantially tubular, linear orientation while within a delivery shaft, but can automatically retain the memory shape of the supporting structure once extended from the delivery shaft. SMMs have a shape memory effect in which they can be made to remember a particular shape. Once a shape has been remembered, the SMM may be bent out of shape or deformed and then returned to its original shape by unloading from strain or heating. Typically, SMMs can be shape memory alloys (“SMA”) comprised of metal alloys, or shape memory plastics (“SMP”) comprised of polymers.

Usually, an SMA can have any non-characteristic initial shape that can then be configured into a memory shape by heating the SMA and conforming the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The main types of SMAs are as follows: copper-zinc-aluminium; copper-aluminium-nickel; nickel-titanium (“NiTi”) alloys known as nitinol; and cobalt-chromium-nickel alloys or cobalt-chromium-nickel-molybdenum alloys known as elgiloy. Typically, the nitinol and elgiloy alloys can be more expensive, but have superior mechanical characteristics in comparison with the copper-based SMAs. The temperatures at which the SMA changes it's crystallographic structure are characteristic of the alloy, and can be tuned by varying the elemental ratios.

For example, the primary material of a supporting structure can be made of a NiTi alloy that forms superelastic nitinol. In the present case, nitinol materials can be trained to remember a certain shape, straightened in a shaft, catheter, or other tube, and then released from the catheter or tube to return to its trained shape. Also, additional materials can be added to the nitinol depending on the desired characteristic.

An SMP is a shape-shifting plastic that can be fashioned into a supporting structure in accordance with the present invention. Also, it can be beneficial to include at least one layer of an SMA and at least one layer of an SMP to form a multilayered body; however, any appropriate combination of materials can be used to form a multilayered supporting structure. When an SMP encounters a temperature above the lowest melting point of the individual polymers, the blend makes a transition to a rubbery state. The elastic modulus can change more than two orders of magnitude across the transition temperature (“Ttr”). As such, an SMP can be formed into the desired shape of a supporting structure by heating it above the Ttr, fixing the SMP into the new shape, and cooling the material below Ttr. The SMP can then be arranged into a temporary shape by force and then resume the memory shape once the force has been applied. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP can be used in accordance with the present invention.

Additionally, the supporting structure can be comprised of a variety of known suitable deformable materials, including stainless steel, silver, platinum, tantalum, palladium, cobalt-chromium alloys such as L605, MP35N, or MP20N, niobium, iridium, any equivalents thereof, alloys thereof, and combinations thereof The alloy L605 is understood to be a trade name for an alloy available from UTI Corporation of Collegeville, Pa., including about 53% cobalt, 20% chromium and 10% nickel. The alloys MP35N and MP20N are understood to be trade names for alloys of cobalt, nickel, chromium and molybdenum available from Standard Press Steel Co., Jenkintown, Pa. More particularly, MP35N generally includes about 35% cobalt, 35% nickel, 20% chromium, and 10% molybdenum, and MP20N generally includes about 50% cobalt, 20% nickel, 20% chromium and 10% molybdenum.

Additionally, the supporting structure can include a biocompatible material capable of expansion upon exposure to the environment within the body lumen. Examples of such biocompatible materials can include a suitable hydrogel, hydrophilic polymer, biodegradable polymers, and bioabsorbable polymers. Examples of such polymers can include poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like.

Furthermore, the supporting structure can be formed from a ceramic material. In one aspect, the ceramic can be a biocompatible ceramic which optionally can be porous. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like. Optionally, the ceramic can be provided as sinterable particles that are sintered into the shape of an endoprosthesis or layer thereof.

Moreover, the endoprosthesis can include a radiopaque material to increase visibility during placement. Optionally, the radiopaque material can be a layer or coating any portion of the endoprosthesis. The radiopaque materials can be platinum, tungsten, silver, stainless steel, gold, tantalum, bismuth, barium sulfate, or a similar material.

1. Drug-Eluting Endoprosthesis

In one embodiment, the present invention includes a drug-eluting endoprosthesis configured for inhibiting restenosis and for promoting healing of a lesion in a body of a subject. Such an endoprosthesis includes at least a supporting structure and dexamethasone. The supporting structure is configured and dimensioned to be placed in the body of the subject, such as a body lumen. A therapeutically effective amount of dexamethasone is disposed on the supporting structure. The therapeutically effective amount of dexamethasone allows for the drug to elute from the supporting structure so as to obtain a concentration of dexamethasone that is sufficient for inhibiting restenosis and that is substantially devoid of inhibiting cell migration adjacent or proximal to the supporting structure when disposed within the subject such that migrating cells promote healing of the lesion. Moreover, the concentration of dexamethasone can be modulated so as to induce the migration of such cells so as to promote healing of the lesion by re-endothelialization. Optionally, the lesion is in a body lumen selected from the group consisting of a blood vessel, artery, coronary artery, vein, esophageal lumen, and urethra, and such a lesion may be caused by deployment of the endoprosthesis.

In one embodiment, the present invention includes an endoprosthesis that includes a supporting structure having a therapeutically effective amount of dexamethasone that inhibits restenosis and thrombosis. Additionally, the drug-eluting endoprosthesis can reduce the rate of restenosis and/or thrombosis to a level of about 0% to 25%.

Additionally, the dexamethasone can be any pharmaceutically acceptable salt, ester, or prodrug of the dexamethasone. The preparation of pharmaceutically acceptable salts, esters, and/or prodrugs of bioactive agents, such as dexamethasone, is well known in the art.

In one embodiment, the dexamethasone can be in the form of a derivative of dexamethasone. A derivative can be prepared by making minor substitutions such as hydroxylating, methylating, ethylating, or otherwise minimally altering a substitutent. However, any derivative of dexamethasone in accordance with the present invention should have the property of inhibiting restenosis and thrombosis while not inhibiting cell migration or by inducing cell migration as described herein.

The dexamethasone can be present on the medical device in a variety of amounts and concentrations. One well-known unit of measuring the amount of a drug on a medical device is the amount (e.g., weight or moles) per length of the medical device. As such, an endoprosthesis in accordance with the present invention can include dexamethasone at about 10 ng/mm to about 10 mg/mm of endoprosthesis length. More preferably, dexamethasone can be present at about 100 ng/mm to about 1 mg/mm of endoprosthesis length. Even more preferably, dexamethasone can be present at about 1 ug/mm to about 100 ug/mm of endoprosthesis length. Still more preferably, dexamethasone can be present from about 5 ug/mm to about 50 ug/mm of endoprosthesis length. Still more preferably, dexamethasone can be present from about 8 ug/mm to about 25 ug/mm of endoprosthesis length. Most preferably, dexamethasone can be present at about 10 ug/mm of endoprosthesis length. For example, dexamethasone can be present at 10 ug/mm of stent length.

In one embodiment, the amount of dexamethasone on an endoprosthesis can be described as the total amount of dexamethasone. As such, an endoprosthesis in accordance with the present invention can include about 10 ng to about 10 mg of dexamethasone. More preferably, dexamethasone can be present at about 100 ng to about 1 mg. Even more preferably, dexamethasone can be present at about 1 ug to about 500 ug. Still more preferably, dexamethasone can be present at about 10 ug to about 250 ug. Still more preferably, dexamethasone can be present at about 100 ug to about 200 ug. Most preferably, dexamethasone can be present at about 150 ug.

In one embodiment, dexamethasone can be included in a carrier, such as a polymeric coating as described in more detail below. As such, an endoprosthesis in accordance with the present invention can include dexamethasone at a concentration of about 10 ng/ml to about 10 mg/ml. More preferably, dexamethasone can be present at a concentration of about 100 ng/ml to about 1 mg/ml. Even more preferably, dexamethasone can be present at a concentration of about 1 ug/ml to about 100 ug/ml. Still more preferably, dexamethasone can be present at a concentration of about 5 ug/ml to about 50 ug/ml. Still more preferably, dexamethasone can be present at a concentration of about 8 ug/ml to about 25 ug/ml. Most preferably, dexamethasone can be present at a concentration of about 10 ug/ml.

In one embodiment, dexamethasone can be included on the endoprosthesis in an amount that generates a local concentration of the analog in the tissues, cells, cellular matrices, body fluids, blood, or the like adjacent or proximal to the endoprosthesis. As such, an endoprosthesis in accordance with the present invention can produce a local concentration of dexamethasone at about 10 pg/ml to about 10 mg/ml. More preferably, the endoprosthesis can produce a local concentration of dexamethasone at about 100 pg/ml to about 1 mg/ml. Even more preferably the endoprosthesis can produce a local concentration of dexamethasone at about 1 ng/ml to about 100 ug/ml. Still more preferably, the endoprosthesis can produce a local concentration of dexamethasone at about 10 ng/ml to about 10 ug/ml. Still more preferably, the endoprothesis can produce a local concentration of dexamethasone at about 100 ng/ml to about 1 ug/ml. Most preferably, the endoprosthesis can produce a local concentration of dexamethasone at about 500 ug/ml.

In one embodiment, dexamethasone can be included on the endoprosthesis in an amount that generates a sustained local concentration of the analog in the tissues, cells, cellular matrices, body fluids, blood, or the like adjacent or proximal to the endoprosthesis that is expressed as molarity. As such, an endoprosthesis in accordance with the present invention can produce a sustained local concentration of dexamethasone at about 10 pM to about 10 mM. More preferably, the endoprosthesis can produce a sustained local concentration of dexamethasone at about 100 pM to about 1 mM. Even more preferably, the endoprosthesis can produce a sustained local concentration of dexamethasone at about 1 nM to about 100 uM. Still more preferably, endoprosthesis can produce a sustained local concentration of dexamethasone at about 10 nM to about 10 uM. Still more preferably, the endoprothesis can produce a sustained local concentration of dexamethasone at about 100 nM to about 1 uM. Most preferably, the endoprosthesis can produce a sustained local concentration of dexamethasone at about 300 nM.

2. Dexamethasone Formulations

In one embodiment, the endoprosthesis includes a pharmaceutically acceptable carrier containing the dexamethasone disposed on the supporting structure. The pharmaceutically acceptable carrier can be any carrier known in the field of pharmaceutics that can be applied and retained on a supporting structure of an endoprosthesis.

In one embodiment, the endoprosthesis includes a carrier configured as a coating disposed on the supporting structure that contains dexamethasone. Optionally, the coating is a biocompatible polymer. In another option, the coating controls the rate dexamethasone is eluted from the supporting structure. Coatings that are suitable for use in this invention include, but are not limited to, polymeric coatings that can comprise any polymeric material in which the drug can be contained. The coating can be hydrophilic, hydrophobic, biodegradable, non-biodegradable, and combinations thereof.

The polymeric coating for use in the coating or the body of the endoprosthesis can be selected from the group consisting of polycarboxylic acids, cellulosic polymers, gelatin, polyvinylpyrrolidone, maleic anhydride polymers, polyamides, polyvinyl alcohols, polyethylene oxides, glycosaminoglycans, polysaccharides, polyesters, polyurethanes, silicones, polyorthoesters, polyanhydrides, polycarbonates, polypropylenes, polylactic acids, polyglycolic acids, polycaprolactones, polyhydroxybutyrates, polyacrylamides, polyethers, mixtures thereof, derivatives thereof, copolymers thereof, and other like polymers. Coatings prepared from polymeric dispersions such as polyurethane dispersions (BAYHYDROL, etc.) and acrylic acid latex dispersions can also be used with the therapeutic agents of the present invention.

The biodegradable polymers that can be used in the coating or the body of the endoprosthesis can be selected from the group consisting of poly(L-lactic acids), poly(DL-lactic acids), polycaprolactones, polyhydroxybutyrates, polyglycolides, poly(diaxanones), poly(hydroxy valerates), polyorthoesters, poly(lactide-co-glycolides), polyhydroxy(butyrate-co-valerates), polyglycolide-co-trimethylene carbonates, polyanhydrides, polyphosphoesters, polyphosphoester-urethanes, polyamino acids, polycyanoacrylates, biomolecules, fibrin, fibrinogen, cellulose, starch, collagen, hyaluronic acid, mixtures thereof, derivatives thereof, copolymers thereof, and like polymers.

The biostable polymers that can be used in the coating or the body of the endoprosthesis can be selected from the group consisting of polyurethanes, silicones, polyesters, polyolefins, polyamides, polycaprolactams, polyimides, polyvinyl chlorides, polyvinyl methyl ethers, polyvinyl alcohols, acrylic polymers, polyacrylonitriles, polystyrenes, vinyl polymers, polymers including olefins (e.g., styrene acrylonitrile copolymers, ethylene methyl methacrylate copolymers, ethylene vinyl acetate, and other like polymers), polyethers, rayons, cellulosics (e.g., cellulose acetate, cellulose nitrate, cellulose propionate, and other like polymers), parylene, mixtures thereof, derivatives thereof, copolymers thereof, and like polymers.

Additionally, various other polymers can be used in the coating or body of the endoprosthesis. An example of such a polymer is poly(MPC_{w}:LAMₓ:HPMA_{y}:TSMA_{z}) where w, x, y, and z represent the molar ratios of monomers used in the feed for preparing the polymer and MPC represents the unit 2-methacryoyloxyethylphosphorylcholine, LMA represents the unit lauryl methacrylate, HPMA represents the unit 2-hydroxypropyl methacrylate, and TSMA represents the unit 3-trimethoxysilylpropyl methacrylate.

The dexamethasone described herein can be applied to the endoprosthesis. For example, the dexamethasone can be incorporated into the coating applied to the endoprosthesis. Alternatively, the dexamethasone can be impregnated within the body of the endoprosthesis. Incorporation of the dexamethasone into a polymeric coating or body of an endoprosthesis can be carried out by dipping the polymer-coated endoprosthesis or absorptive endoprosthesis into a solution containing the dexamethasone for a sufficient period of time (such as, for example, five minutes) and then drying the endoprosthesis for a sufficient period of time (e.g., 10, 15, or 30 minutes). The endoprosthesis including the dexamethasone can be delivered into a blood vessel, such as the coronary vessel, by well-known means of endoprosthsis deployment. For example, a stent can be delivered with a balloon catheter.

The dexamethasone can be formulated into a variety of pharmaceutically acceptable formulations that can be included with the endoprosthesis, and/or can be prepared for application of the dexamethasone to the endoprosthsis. This can include formulating the dexamethasone with a pharmaceutically acceptable carrier, which is a non-toxic solid, semi-solid or liquid filler, diluent, encapsulating material or formulation auxiliary of any type. The compositions that can be included with the endoprosthesis can be comprised of pharmaceutically acceptable sterile aqueous or nonaqueous solutions, dispersions, suspensions or emulsions, as well as sterile powders for reconstitution into sterile solutions or dispersions just prior to be included with the endoprosthesis. Examples of suitable aqueous and nonaqueous carriers, diluents, solvents or vehicles include water, ethanol, polyols (e.g., glycerol, propylene glycol, polyethylene glycol, and the like), carboxymethylcellulose and suitable mixtures thereof, vegetable oils (e.g., olive oil), organic esters such as ethyl oleate, and the like. The proper characteristic of the composition including the dexamethasone can be maintained by the use of coating materials such as lecithin, by the maintenance of the required particle size in the case of dispersions, and by the use of surfactants in the formulation.

The compositions containing dexamethasone that can be included with the endoprosthesis may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin.

In some cases, in order to prolong the effect of the drug, it is desirable to slow the absorption of the drug from the endoprosthesis. This may be accomplished by the use of crystalline or amorphous materials with poor water solubility. The rate of absorption of the drug can depend upon its rate of dissolution which, in turn, may depend upon crystal size and crystalline form. Alternatively, delayed absorption of the dexamethasone from the endoprosthesis can be accomplished by dissolving or suspending the drug in an oil or hydrophobic carrier prior to being included with the endoprosthesis.

Additionally, the dexamethasone can be formulated into microencapsule matrices before being included with the endoprosthesis. As such, the dexamethasone can be included within a biodegradable polymer, such as polylactide-polyglycolide. Depending upon the ratio of drug to polymer and the nature of the particular polymer employed, the rate of drug release can be controlled. Similarly, the dexamethasone can be included in liposomes or microemulsions which are compatible with body tissues.

Also, the dexamethasone can be mixed with inert, pharmaceutically acceptable materials such as any of the following: excipients or carriers such as sodium citrate or dicalcium phosphate; fillers or extenders such as starches, lactose, sucrose, glucose, mannitol, and silicic acid; binders such as, for example, carboxymethylcellulose, alginates, gelatin, polyvinylpyrrolidone, sucrose, and acacia; humectants such as glycerol; disintegrating agents such as agar, calcium carbonate, starch, alginic acid, certain silicates, and sodium carbonate; solution retarding agents such as paraffin; absorption accelerators such as quaternary ammonium compounds; wetting agents such as cetyl alcohol and glycerol monostearate; absorbents such as kaolin and bentonite clay; and lubricants such as talc, calcium stearate, magnesium stearate, solid polyethylene glycols, and sodium lauryl sulfate.

Additionally, the dexamethasone can be included in a liquid form that can be absorbed into the coating or body of the endoprosthesis. Such liquid forms can be pharmaceutically acceptable emulsions, solutions, suspensions, syrups, and the like. In addition to the dexamethasone, the liquids may contain inert diluents commonly used in the art such as water, other solvents, solubilizing agents, emulsifiers, ethyl alcohol, isopropyl alcohol, ethyl carbonate, ethyl acetate, benzyl alcohol, benzyl benzoate, propylene glycol, 1,3-butylene glycol, dimethyl formamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof

III. Delivering Dexamethasone

In one embodiment, the present invention includes a method of delivering dexamethasone into the body of a subject. Such a method of delivering dexamethasone can be used for inhibiting restenosis and promoting healing of a lesion in a body of a subject. For example, the lesion can be healed by dexamethasone that does not substantially inhibit the migration of smooth muscle cells and/or endothelial cells into the lesion. While an amount of dexamethasone may somewhat reduce the migration of some cells, the migration is allowed to proceed so as to be capable of promoting healing of the lesion. This can be particularly advantageous for lesions that are caused by the deployment of an endoprosthesis. By promoting healing of the lesion, thrombosis is less likely to occur. That is, re-endothelialization of the lesion can significantly reduce the onset of thrombosis because clot forming materials can be covered by the migrating cells.

Additionally, a method of delivering dexamethasone can be used for inhibiting restenosis and inducing re-endothelialization of a lesion. For example, the lesion can be healed by an amount of dexamethasone that induces the migration of endothelial cells into the lesion, which promotes healing of the lesion. This can be particularly advantageous for lesions that are caused by the deployment of an endoprosthesis. By inducing cell migration and promoting healing of the lesion, thrombosis is less likely to occur. That is, re-endothelialization of the lesion by inducing cell migration can significantly reduce the onset of thrombosis because clot forming materials can be covered by the migrating cells.

Accordingly, one method includes deploying an endoprosthesis that contains dexamethasone into the body of the subject. The endoprosthesis includes a supporting structure configured and dimensioned to be placed in the body of the subject. A therapeutically effective amount of dexamethasone is disposed on the supporting structure. The dexamethasone is eluted from the supporting structure so as to obtain a concentration of dexamethasone in the body and distal, adjacent, or proximal to the supporting structure that is sufficient for inhibiting restenosis and that is substantially devoid of inhibiting cell migration adjacent to the supporting structure such that migrating cells promote healing of the lesion. Moreover, the concentration of dexamethasone in the body and adjacent or proximal to the supporting structure is sufficient for inducing the migration of such cells. Optionally, the lesion is in a body lumen selected from the group consisting of a blood vessel, artery, coronary artery, vein, esophageal lumen, and urethra.

In one embodiment, the present invention includes a method of inhibiting restenosis and thrombosis in a body of a subject. Such a method includes deploying an endoprosthesis that contains dexamethasone into the body of the subject. The endoprosthesis includes a supporting structure configured and dimensioned to be placed in the body of the subject. A therapeutically effective amount of dexamethasone is disposed on the supporting structure. The dexamethasone is eluted from the supporting structure so as to obtain a concentration of dexamethasone in the body and adjacent to the supporting structure that is sufficient for inhibiting restenosis and thrombosis.

In one embodiment, the present invention includes a method of inhibiting restenosis and thrombosis while promoting healing of a lesion in a body of a subject. Such a method includes delivering dexamethasone into the body of the subject that is susceptible to restenosis and/or thrombosis. In part, this can be achieved by delivering a therapeutically effective amount of dexamethasone. The delivery of dexamethasone can be configured and modulated in order to obtain a concentration of dexamethasone in the body that is sufficient for inducing migration of cells, such as endothelial and/or smooth muscle cells.

When used in the above or other treatments, a therapeutically effective amount of one of the compounds of the present invention may be employed in pure form or, where such forms exist, in pharmaceutically acceptable salt, ester, or prodrug form. Alternatively, the compound may be administered as a pharmaceutical composition containing the compound of interest in combination with one or more pharmaceutically acceptable excipients. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration; and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

In one embodiment, dexamethasone can be delivered in an amount that generates a local concentration of dexamethasone in the tissues, cells, cellular matrices, body fluids, blood, or the like distal, adjacent, or proximal to the endoprosthesis. This can include achieving a concentration that inhibits restenosis and thrombosis without inhibiting cell migration. As such, dexamethasone can be delivered to produce a local concentration of about 10 pg/ml to about 10 mg/ml. More preferably, the dexamethasone can be delivered to produce a local concentration of about 100 pg/ml to about 1 mg/ml. Even more preferably, the dexamethasone can be delivered to produce a local concentration of about 1 ng/ml to about 100 ug/ml. Still more preferably, the dexamethasone can be delivered to produce a local concentration of about 10 ng/ml to about 10 ug/ml. Still more preferably, the dexamethasone can be delivered to produce a local concentration of about 100 ng/ml to about 1 ug/ml. Most preferably, the dexamethasone can be delivered to produce a local concentration of at about 500 ug/ml.

In one embodiment, the dexamethasone can be delivered in an amount that generates a sustained local concentration of the analog in the tissues, cells, cellular matrices, body fluids, blood, or the like proximate to the endoprosthesis that is expressed as molarity. As such, delivery of the dexamethasone can produce a sustained local concentration of about 10 pM to about 10 mM. More preferably, dexamethasone can be delivered to produce a sustained local concentration of about 100 pM to about 1 mM. Even more preferably, the dexamethasone can be delivered to produce a sustained local concentration of about 1 nM to about 100 uM. Still more preferably, the dexamethasone can be delivered to produce a sustained local concentration of about 10 nM to about 10 uM. Still more preferably, the dexamethasone can be delivered to produce a sustained local concentration about 100 nM to about 1 uM. Most preferably, the dexamethasone can be delivered to produce a sustained local concentration at about 300 nM.

The total daily dose of the compounds of this invention administered to a human or lower animal may range from about 0.01 to about 10 mg/kg/day. For the purposes of local delivery from a stent, the daily dose that a patient will receive depends on the length of the stent. For example, a 15 mm coronary stent may contain a drug in an amount ranging from about 1 to about 120 mg and may deliver that drug over a time period ranging from several hours to several weeks.

In one embodiment, the method includes eluting the dexamethasone from a pharmaceutically acceptable carrier, such as a polymeric coating, disposed on the supporting structure. As such, the coating can be any of the carriers described herein and well known in the art. In some instances, the carrier can be configured to modulate the rate of elution of the dexamethasone, which can include a substantially constant or steady-state rate. Also, this can include an initial burst followed by 0, 1st, or 2nd order delivery kinetics.

In one embodiment, the method includes inhibiting restenosis of a body lumen adjacent to the supporting structure. This can also be performed while promoting healing of the lesion in a wall of the body lumen that is caused by the supporting structure. The healing of the lesion is promoted by allowing and/or inducing migration of endothelial cells and/or smooth muscle cells to the lesion to be substantially uninhibited. Such cell migration can lead to re-endothelialization of the lesion.

Western blots have shown the levels of specific proteins using antibodies to those proteins in response to dexamethasone. The results show the effects of anti-restenotic agents on phosphorylation of the mTOR effector p70S6K in human coronary artery endothelial cells in vitro. It has been shown that paclitaxel and zotarolimus inhibit the activation of p70S6K. On the other hand, it has been shown that dexamethasone does not inhibit the activation of p70S6K.

Additionally, it has been shown that paclitaxel, but not zotarolimus or dexamethasone, inhibits migration of human coronary artery endothelial cells in vitro. Paclitaxel block migration and dexamethasone and mTOR agents do not block migration; however both paclitaxel and mTOR agents reduce p70S6K(T389) phosphorylation. Zotarolimus reduces pRb phosphorylation, consistent with the ability of zotarolimus to inhibit hEC proliferation. These results suggest that in the presence of multiple growth factors and fetal bovine serum (FBS), inhibition of p70S6K alone is insufficient to reduce migration. Dexamethasone and zotarolimus are not predicted to substantially inhibit re-endothelialization of vascular legions. Also, dexamethasone promotes re-endothelialization of vascular lesions by inducing cell migration. In contrast, paclitaxel may inhibit re-endothelialization by blocking endothelial cell and/or smooth muscle cell migration.

### EXAMPLES

Previously, it has been found that drug-eluting stents have reduced restenosis rates, but elute drugs that interfere with endothelial cell (EC) and/or smooth muscle (SMC) migration and re-endothelialization of lesions, which leads to thrombosis. More particularly, when drug-eluting stents are used in humans, the eluted drugs interfere with cell migration and re-endothelialization of lesions, and thereby increasing the chance of thrombosis. As such, experiments have been conducted in order to determine the effects of the anti-restenotic agents zotarolimus, sirolimus, paclitaxel, and dexamethasone on migration and on phosphorylation of p70S6K, an intracellular molecule which has been reported to mediate the effects of S1P-1 mediated cell migration. The phosphorylated p70S6k is one regulator of cell growth and proliferation, and can be studied so that the effects of the anti-restenotic agents can be determined with respect to the roles of phosphorylated p70S6K in the migration of cells.

In addition to their known anti-proliferative effects it has been proposed that some mTOR antagonists and paclitaxel also prevent the migration of smooth muscle into the developing neointima and that this contributes to their anti-restenotic efficacy. It has been reported that the rapamycin analog sirolimus inhibits the migration of rat vascular smooth muscle cells in response to sphingosine-1-phosphate (S1P) and migration induced by platelet-derived growth factor homodimer B (PDGF-BB). The mechanisms by which mTOR antagonists (i.e., sirolimus, zotarolimus, or everolimus) exert this activity has been reported to involve inhibition of p70S6K. According to this hypothesis, mTOR antagonists inhibit the phosphorylation and activation of p70S6K resulting in a reduction of free elongation initiation factor 4F (eIF-4F) and inhibition of protein translation. The reduction in proteins critical to cell cycle progression lead to cell cycle arrest at the G₁ to S phase transition in various cell types including vascular smooth muscle and in the inhibition of migration.

The cancer chemotherapeutic, paclitaxel is hypothesized to directly interfere with microtubule homeostasis leading to the inhibition of EC migration and proliferation, which can lead to increased thrombosis. To examine the comparative effects of sirolimus, zotarolimus, dexamethasone, and paclitaxel on human coronary artery smooth muscle and endothelial cell migration, studies were conducted using modified Boyden chambers. As such, EC and SMC migration in response to hFGF, HEGF, VEGF, R³-IGF-1 and FBS or FBS alone was determined using a modified Boyden chamber. Migration was induced using a variety of chemotactic stimuli including fetal bovine serum (FBS) in the presence and absence of growth factors as well as platelet-dependent growth factor homodimer B (PDGF-BB). Anti-migratory activity was assessed after acute drug treatment and after pretreatment (24 hours) in basal or growth conditions. Surprisingly these data show that reduced p70S6K activation alone will not result in anti-migratory activity when exposed to multiple chemotactic agents as is likely to occur in vivo.

I. Maintenance of Cell Lines

Primary human coronary artery smooth muscle (hCaSMC) and endothelial cells (hCaEC) obtained from Cambrex (Walkersville, Md.) were routinely maintained in media recommended by the manufacturer. The hCaSMC were grown in a smooth muscle growth medium (“minimal media”, SmnBM, Cambrex) supplemented with the appropriate SingleQuot® to yield the complete medium containing 5% FBS, 0.1% hEGF, 0.2% hFGF-B, and 0.1% insulin (hCaSMC growth media). The hCaEC were maintained in the corresponding complete medium consisting of an endothelial cell growth medium (“minimal media”, EBM-2, Cambrex) supplemented with the appropriate SingleQuot® to yield the complete medium containing 5% FBS, 0.1% HEGF-B, 0.4% FGF, 0.1% R3-IGF, 0.04% hydrocortisone, 0.1% VEGF, and 0.1% ascorbic acid (hCaEC growth media). Cells were routinely propagated in the appropriate medium in humidified incubators maintained at 37° C. in an atmosphere containing 5% CO₂. When necessary, each cell type was passed at ˜75% confluence.

Cells were maintained and used prior to 7 population doublings in all experiments. To expand cells prior to assay, frozen hCaSMC or hCaEC cells were thawed and cultured in T-162 flasks in smooth muscle growth medium (SmGM) or endothelial cell growth medium-2 MV (EGM-2 MV), respectively, with the appropriate supplements. The cell medium was changed the next day and then every other day until cells reached 70-90% confluency. Growth medium was removed and cells were rinsed twice with PBS (without Ca²⁺ and Mg²⁺). To detach cells, sufficient trypsin/EDTA (3 ml) was added to cover the cell monolayer and the flask incubated at room temperature. After cells detached, trypsin was neutralized by addition of 6 ml complete medium. Cells were counted, transferred to a 50 ml tube and centrifuged. The cell pellet was resuspended in growth medium to the appropriate cell density for experiments.

II. Migration Experiment

Cells (hCaEC or hCaSMC) were synchronized for 24 hours in basal media prior to all migration experiments. In acute studies, cells were collected, counted, and 200,000 hCaEC or 160,000 hCaSMC placed in the upper chambers of modified boyden chambers in 24 well plates (BD Biosciences). Plates contained microporous membranes of 3.0 and 8.0 um for hCaEC and hCaSMC, respectively. Chambers used in hCaEC migration experiments were coated with fibronectin (BD BioCoat). In pretreatment experiments, cells were synchronized in the presence of drug or treated for 24 hours in growth media after synchronization but prior to cell counting and migration. All cells were resuspended in basal media containing drugs or solvent (control) prior to migration. Cells were allowed to migrate in response to chemotatic agents for 22-24 hours. After migration, cells were stained with calcein-AM for 90 minutes and fluorescence determined using a plate reader capable of measuring fluorescence emitted by the bottom of the microporous filter. Fluorescence associated with migration in response to basal media was used to correct for background. Data are expressed as percent or fold of control fluorescence.

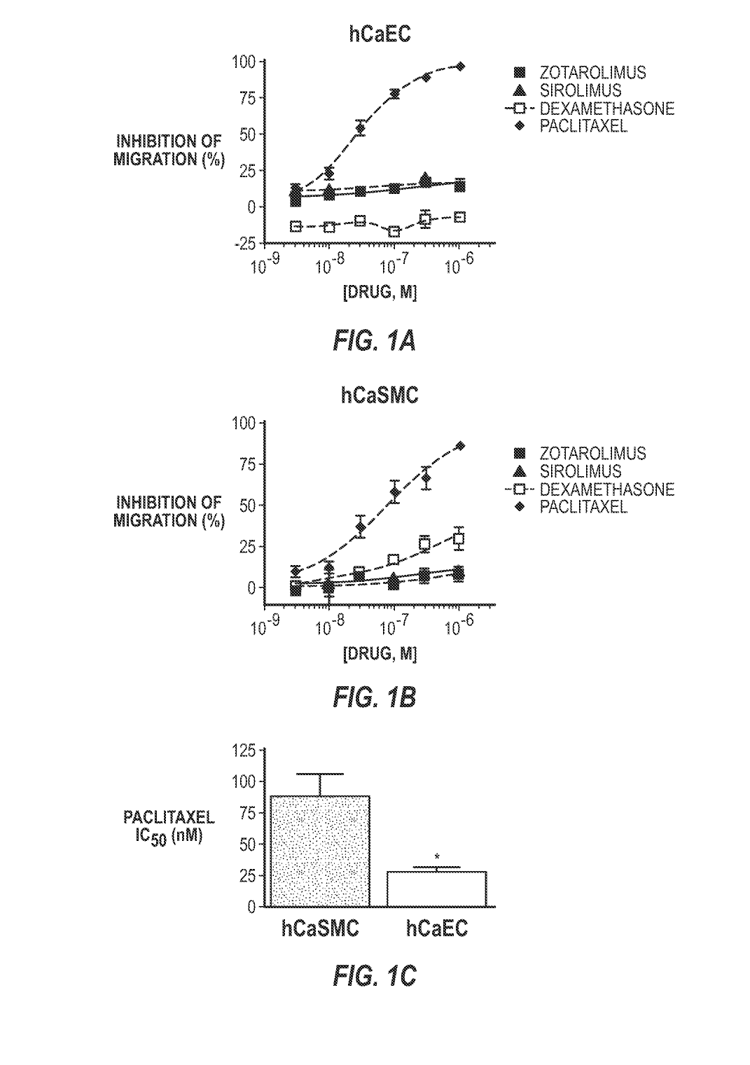

FIGS. 1A-1C illustrate the results of acute drug exposure experiments. FIG. 1A is a graph illustrating the effect of mTOR antagonists (zotarolimus and sirolimus) dexamethasone, and paclitaxel on inhibiting migration of human coronary artery endothelial cells. FIG. 1B is a graph illustrating the effect of zotarolimus, sirolimus, dexamethasone, and paclitaxel on inhibiting migration of human coronary artery smooth muscle cells. FIG. 1C is a graph illustrating the IC₅₀ of paclitaxel on human coronary artery smooth muscle cells and human coronary artery endothelial cells. Cells were synchronized for 24 hours prior to the addition of drug. Cells were not pretreated prior to the induction of migration. Migration was induced by 5% fetal bovine serum (FBS) and the cells were exposed to agents during the 22 hour migration period. Paclitaxel inhibits hCaEC migration with an IC₅₀ of 28.2±3.4 nM and a maximal efficacy of 96.8±1.8% (FIG. 1C, mean±SEM, n=3). The mTOR antagonists sirolimus and zotarolimus, had no effect on hCaEC migration and dexamethasone appeared to exert a modest pro-migratory effect (FIG. 1A). In hCaSMC paclitaxel also exerts a significant anti-migratory effect with an IC₅₀ of 87.4±18 nM and a maximal efficacy of 86.9±2.9% (FIG. 1B). Paclitaxel demonstrates a significantly greater anti-migratory potency in hCaEC compared to hCaSMC (FIG. 1C). Dexamethasone has a small inhibitory effect on hCaSMC migration induced by FBS (FIG. 1B). As such, paclitaxel is a potent antimigratory agent, dexamethasone enhances hCaEC migration but is slightly inhibitory in hCaSMC.

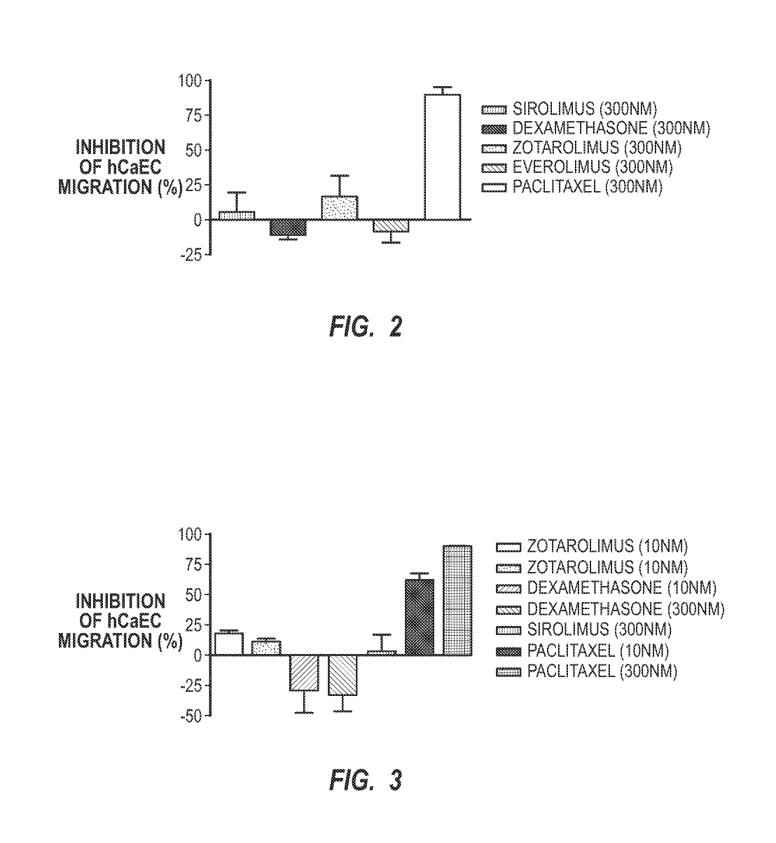

To determine if pre-exposure with drugs was required to demonstrate anti-migratory activity, following synchronization, hCaEC were pretreated with agents in hCaEC full growth media for 24 hours. Migration of hCaEC was induced by full growth media and the effects of the drugs sirolimus, dexamethasone, zotarolimus, everolimus, and paclitaxel on hCaEC migration determined. Following pretreatment cells were counted and re-suspended in basal media containing drugs and migration initiated. The graph in FIG. 2 shows that only paclitaxel inhibits full growth media induced hCaEC migration after drug pretreatment.

To determine if growth factors contained in full growth media played a role in the inability of the mTOR antagonists (sirolimus or zotarolimus) or dexamethasone to inhibit migration, additional experiments were conducted to determine if drug pretreatment would affect migration induced by 5% FBS in the absence of growth factors. Migration in response to FBS following pretreatment of hCaEC with different concentrations of drugs was determined for the drugs sirolimus, dexamethasone, zotarolimus, and paclitaxel. To explore this effect in hCaEC, cells were synchronized, pretreated with drug in growth media for 24 hours, resuspended in basal media containing drug and migration in response to FBS determined. The results are given in FIG. 3. The mTOR antagonists sirolimus, and zotarolimus did not significantly block hCaEC migration. Dexamethasone promoted hCaEC migration. Paclitaxel significantly blocks migration.

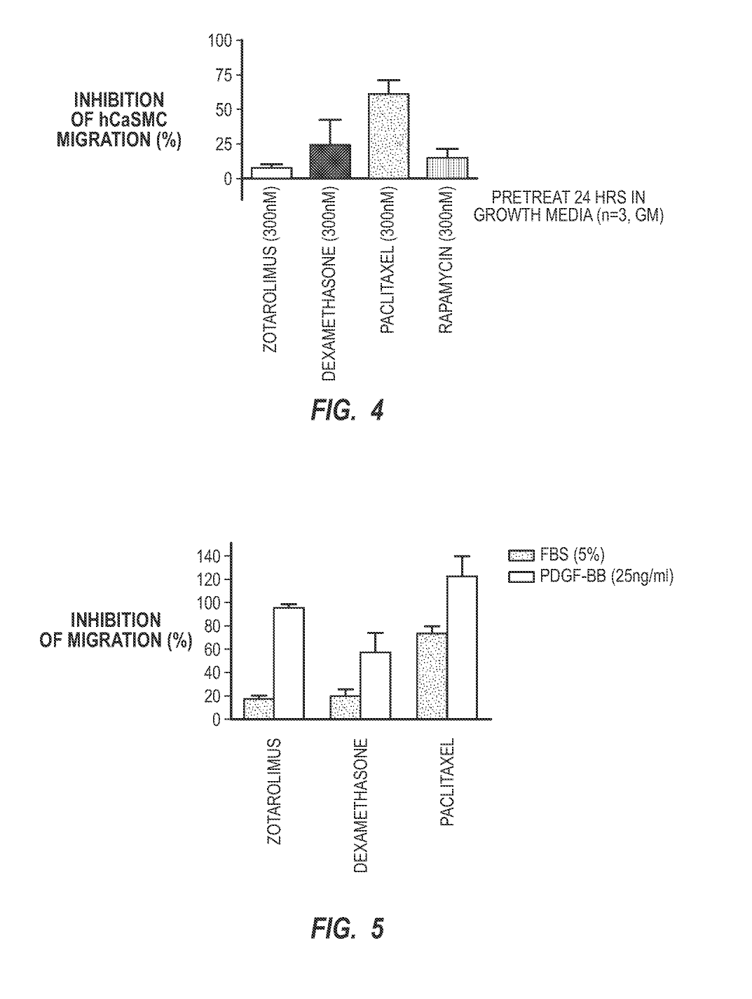

Another experiment was conducted to determine if growth factors contained in full growth media played a role in the inability of the mTOR antagonist zotarolimus or dexamethasone to inhibit human coronary artery smooth muscle cell (hCaSMC) migration. Migration in response to FBS following pretreatment of hCaSMC with different concentrations of drugs was determined for the drugs dexamethasone, zotarolimus, rapamycin, and paclitaxel. To explore this effect, hCaSMC were synchronized, pretreated with drug in growth media for 24 hours, resuspended in basal media containing drug and migration in response to FBS alone determined. The results are given in FIG. 4. Similar to their effects in hCaEC the mTOR antagonists rapamycin and zotarolimus did not significantly block hCaSMC migration. Dexamethasone exerts a small anti-migratory effect in hCaSMC and paclitaxel significantly blocks migration.

Unexpectedly, the foregoing results show that pre-exposing hCaEC or hCaSMC with rapamycin or zotarolimus for 24 hours followed by additional drug exposure for 22-24 hours had no effect on the migration response induced by FBS or full growth media.

Pre-exposure with the mTOR agent sirolimus has been reported to block rat aortic smooth muscle cell migration in response to S1P-1 and porcine aortic smooth muscle in response to PDGF-BB. FIG. 5 shows the effects of dexamethasone, zotarolimus, and paclitaxel pretreatment for 24 hours on PDGF-BB and serum-induced hCaSMC migration, which were determined in paired experiments. In contrast to their inability to block serum-induced migration, zotarolimus, dexamethasone, and paclitaxel all inhibited migration in response to the single chemotactic agent PDGF-BB (25 ng/ml).

III. Western Blot Assay

Human Coronary Endothelial Cells (hCaEC, Cambrex) were seeded on 100 mm plastic dishes (1-3×10⁵ cells per dish) and incubated at 37° C. with 5% CO₂ in complete growth media containing hFGF, HEGF, VEGF, R³-IGF-1 and 5% FBS (GM). Cells were allowed to grow until dishes were 30-40% confluent. Cells were synchronized by incubation in basal media without supplements (BM) for 24 h. After synchronization the BM was replaced with GM containing test compounds, DMSO and BSA (Experimental dish: GM containing 300 nM test compounds, 0.1% BSA; Positive control: GM containing 0.1% BSA and 0.1% DMSO; Negative control: BM containing 0.1 or 0.5% FBS, 0.1% BSA and 0.1% DMSO).

Cells were incubated for 24 or 36 hours at 37° C. with 5% CO₂. After which dishes were 50-70% confluent. Dishes were rinsed with warm PBS to remove excess FBS and than placed on ice. Whole cell lysates were prepared by adding 150-200 ul of RIPA lysis buffer at 4° C. (50 mM Tris HCl pH 8, 150 mM NaCl, 1% NP-40, 0.5% sodium deoxycholate, 0.1% SDS, 2 mM PMSF, 1 mM sodium orthovanadate, and protease inhibitor cocktail, Santa Cruz Biotechnology) to the culture dishes and collected by scrapping the plate surface with a cell scrapper. Lysates were transferred to 1 ml eppendorf tubes and incubated on ice with vortexing for 30 min. Samples were than centrifuged for 8 min at 10000 rpm. Supernatants were collected and transferred into new tubes and stored at −70° C.

The total protein concentration of the samples was determined by a modification of the method of Bradford (Quick Start Bradford Microplate Standard Assay, Bio-Rad) with bovine γ-globulin as a standard.

Total volume of the loaded sample was 45 ul containing 15 ul of 3× sample buffer (Laemmli Sample Buffer, Bio Rad), and sample (30 ul) containing 25 to 50 ug of protein. Samples were diluted to the same protein concentration. Samples were mixed and boiled at 98° C. for 3-4 minutes. Denaturated samples were separated in 4-15% gradient or 12% Ready Tris-HCl gels (Bio-Rad) in running buffer 25 mM Tris, pH 8.3, 192 mM glycine, 0.1% SDS at 150-180 V for 60-90 minutes and transferred to Hybond-ECL membrane (Amersham Biosciences) in 25 mM Tris, pH 8.3, 192 mM glycine, 20% methanol at 100 V for 1 hour.

Protein transfer to the membrane was verified by staining with Ponceau. Ponceau was removed by washing membranes 3 times for 5 minutes in TBS-T (10 mM Tris, pH 7.4, 150 mM NaCl, and 0.1% Tween 20). The membrane was blocked with a 3% solution of nonfat dry milk (Blotting-Grade Blocker, Bio-Rad) in TBS-T for 1 hour at room temperature. Membranes were then incubated with rabbit anti-phospho-p70S6K (T389) monoclonal antibody (Cell Signaling) 1:200 or 1:400 in 3% nonfat dry milk overnight at +4° C. and gently rocked. Goat polyclonal antibodies raised against the C-terminus of actin of human origin (Santa-Cruz Biotechnology) were used at a 1:800 dilution.

After overnight incubation with the primary antibody, membranes were washed with TBS-T 3 times for 5 minutes followed by incubation with the appropriate horseradish peroxidase linked secondary antibodies in 3% nonfat dry milk according to antibody supplier instructions. After one-hour incubation at room temperature with secondary antibodies the membrane was washed again using TBS-T, 3 times for 5 minutes and developed using ECL Plus (Amersham Biosciences). Images of the membranes were scanned and stored using a multi-channel scanning system FLA-5000 (FUJI Film).

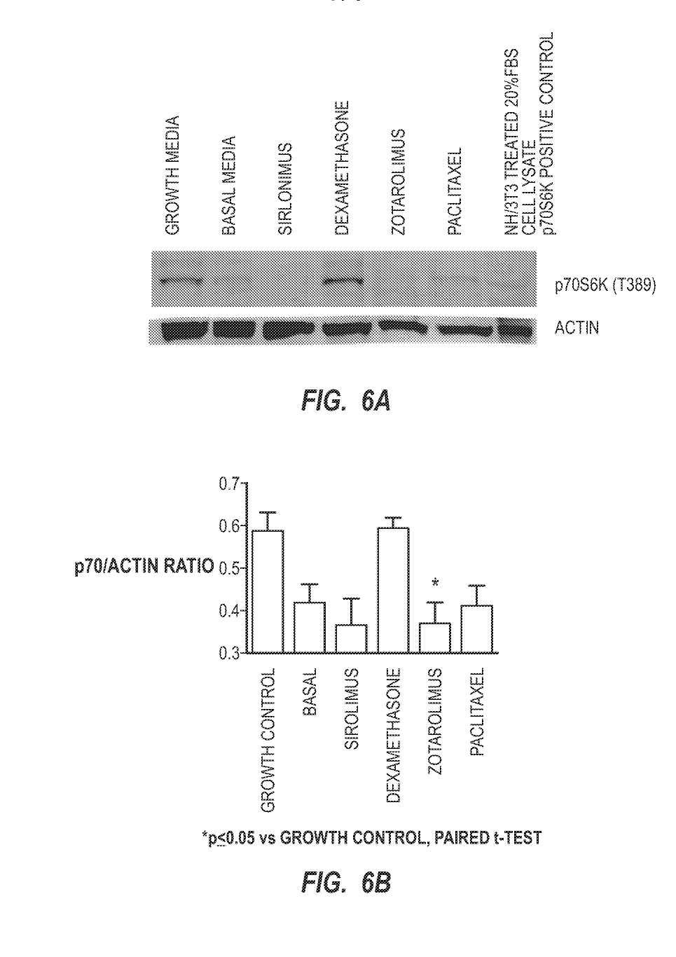

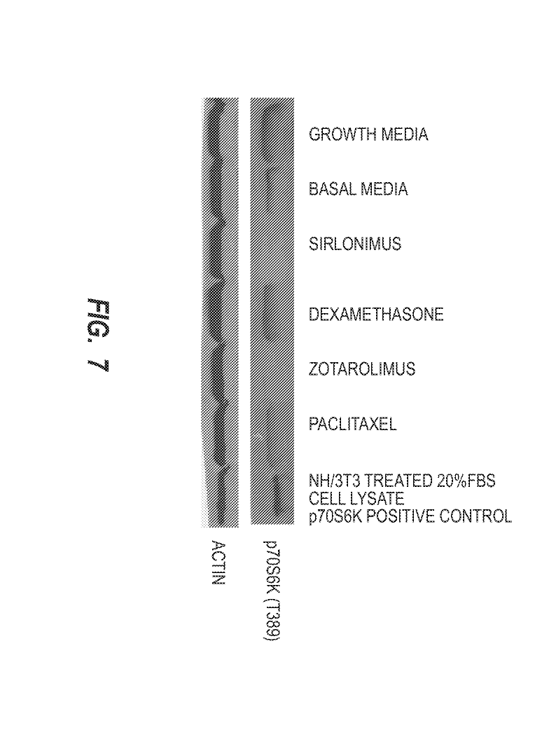

To determine if inhibition of p70S6K was involved in serum-induced migration Western blot experiments were performed on hCaEC and hCaSMC treated using an experimental paradigm similar to those of the migration experiments. Cells were synchronized for 24 hours in basal media followed by drug treatment in growth media for 36 hours. The effects of drug treatment on p70S6K T389 phosphorylation in hCaEC are illustrated in FIG. 6A-6B. It is clear that removal of growth factors and FBS in the basal media results in significant inhibition of p70S6K T389 phosphorylation. Paclitaxel also reduces T389 phosphorylation though this effect is less than that of the mTOR antagonists. Both sirolimus and zotarolimus completely block T389 phosphorylation of p70S6K.

FIG. 6 shows the affect of mTOR antagonists, dexamethasone, and paclitaxel on p70S6K phosphorylation in hCaEC. Dexamethasone slightly augments and mTOR antagonists and paclitaxel inhibit p70S6K phosphorylation in hCaEC. Densitometry of western blots from hCaEC lysates prepared from cells following treatment with drugs (300 nM) or serum-free media (basal control). As shown in FIG. 6B, densitometric analysis results indicate that the effect of zotarolimus results in significant inhibition of the p70S6K T389 to actin ratio compared to growth controls in hCaEC. The western blot in FIG. 6A is illustrative of three independent experiments.

FIG. 7 shows the affect of mTOR antagonists dexamethasone and paclitaxel on p70S6K phosphorylation in hCaSMC. The hCaSMC were synchronized for 24 hours in basal media and incubated in growth media with or without drugs (300 nM) for 36 hours in basal media. Membranes were probed with a rabbit anti-phospho-p70S6K (T389) monoclonal antibody (Cell Signaling). FIGS. 6-7 show that agents produce similar effects on p70S6K phosphorylation at position T389 in both cell types. Again, the mTOR antagonists zotarolimus and sirolimus completely inhibit p70S6K T389 phosphorylation in hCaSMC. Paclitaxel reduces p70S6K, though not as effectively as the mTOR agents, and dexamethasone is without effect. Despite their ability to completely inhibit phosphorylation and activation of p70S6K at T389 the mTOR antagonists do not inhibit migration of either cell type in response to serum. It is concluded that migration induced by a mixture of growth factors and chemokines is not inhibited by mTOR antagonists suggesting that signaling pathways in addition to p70S6K are involved. Furthermore, the anti-migratory activity of the mTOR antagonists is not likely to contribute to their anti-restenotic activity. Conversely, paclitaxel is a potent anti-migratory agent and this effect is likely to contribute to the anti-restenotic activity.

Accordingly, since migration of vascular endothelial cells, such as hCaEC, contribute to vascular wall healing these results suggest that mTOR antagonists would not be expected to significantly interfere with endothelial migration and re-endothelialization of the injured vascular wall. In contrast, these data show that paclitaxel is expected to potently inhibit endothelial migration and re-endothelialization potentially resulting is a delayed healing response. Furthermore these data show that, unexpectedly, dexamethasone has a positive effect on endothelial migration and may promote vascular healing when administered to the lumen of a vessel.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A drug-eluting endoprosthesis for promoting healing of a lesion in a body of a subject, said endoprosthesis comprising:
a supporting structure configured and dimensioned to be placed in the body of the subject; and
a therapeutically effective amount of a dexamethasone disposed on the supporting structure, said therapeutically effective amount of dexamethasone elutes from the supporting structure to obtain a concentration of dexamethasone that is sufficient for inhibiting restenosis and that is substantially devoid of inhibiting cell migration adjacent to the supporting structure when disposed within the subject such that migrating cells promote healing of the lesion.

2. An endoprosthesis as in claim 1, wherein the dexamethasone is a derivative, salt, ester, or prodrug thereof.

3. An endoprosthesis as in claim 1, wherein the therapeutically effective amount of dexamethasone elutes from the supporting structure to obtain a concentration of dexamethasone that is sufficient for inducing cell migration adjacent to the supporting structure.

4. An endoprosthesis as in claim 3, wherein the cells induced to migrate are endothelial cells.

5. An endoprosthesis as in claim 1, further comprising a pharmaceutically acceptable carrier containing dexamethasone being disposed on the supporting structure.

6. An endoprosthesis as in claim 1, further comprising a coating containing dexamethasone being disposed on the supporting structure.

7. An endoprosthesis as in claim 6, wherein the coating is a biocompatible polymer.

8. An endoprosthesis as in claim 7, wherein the biocompatible polymer controls the rate dexamethasone is eluted from the supporting structure.

9. An endoprosthesis as in claim 1, wherein dexamethasone is present on the supporting structure in a concentration from about 10 ng per mm to about 10 mg per mm of endoprosthesis length.

10. An endoprosthesis as in claim 1, wherein the concentration of dexamethasone that is sufficient for inhibiting restenosis and that is substantially devoid of inhibiting cell migration adjacent to the supporting structure when disposed within the subject is from about 10 pg/ml to about 10 mg/ml.

11. An endoprosthesis as in claim 1, wherein dexamethasone elutes from the supporting structure at a rate of about 10 pg/day to about 10 ug/day.

12. An endoprosthesis as in claim 1, wherein the lesion is in a body lumen selected from the group consisting of a blood vessel, artery, coronary artery, vein, esophageal lumen, and urethra.

13. A method of promoting healing of a lesion in a body of a subject, the method comprising:
deploying an endoprosthesis into the body of the subject, said endoprosthesis comprising:
a supporting structure configured and dimensioned to be placed in the body of the subject; and
a therapeutically effective amount of a dexamethasone disposed on the supporting structure; and
eluting dexamethasone from the supporting structure so as to obtain a concentration of dexamethasone in the body and adjacent to the supporting that is sufficient for inhibiting restenosis and that is substantially devoid of inhibiting cell migration adjacent to the supporting structure when disposed within the subject such that migrating cells promote healing of the lesion.

14. A method as in claim 13, wherein the dexamethasone is a derivative, salt, ester, or prodrug thereof.

15. A method as in claim 13, wherein the concentration of dexamethasone is sufficient for inducing cell migration adjacent to the supporting structure.

16. A method as in claim 15, wherein the cells induced to migrate are endothelial cells.

17. A method as in claim 13, further comprising eluting dexamethasone from a pharmaceutically acceptable carrier disposed on the supporting structure.

18. A method as in claim 13, further comprising eluting dexamethasone from a coating disposed on the supporting structure.

19. A method as in claim 18, wherein the coating is a biocompatible polymer.

20. A method as in claim 13, wherein dexamethasone is present on the supporting structure in an amount from about 10 ng per mm to about 10 mg per mm of endoprosthesis length.

21. A method as in claim 13, further comprising achieving a local concentration of dexamethasone adjacent to the endoprosthesis from about 10 pg/ml to about 10 mg/ml.

22. A method as in claim 13, further comprising eluting dexamethasone from the supporting structure at a rate of about 10 pg/day to about 10 ug/day.

23. A method as in claim 13, further comprising positioning the supporting structure in a body lumen selected from the group consisting of a blood vessel, artery, coronary artery, vein, esophageal lumen, and urethra.

24. A method as in claim 13, further comprising inhibiting restenosis of a body lumen adjacent to the supporting structure.

25. A method as in claim 24, further comprising promoting healing of the lesion in a wall of the body lumen that is caused by the supporting structure.

26. A method as in claim 25, wherein the healing of the lesion is promoted by inducing migration of endothelial cells to the lesion.

27. A method as in claim 13, further comprising inhibiting thrombosis adjacent to the endoprosthesis.
